(19) United States
(12) Patent Application Publication
(10) Pub. No.: US 2003/0177531 A1
(43) Pub. Date: Sep. 18, 2003

(54) Regulating metabolism by modifying the level of trehalose-6-phosphate by inhibiting endogenous trehalase levels

(75) Inventors: Oscar Johannes Maria Goddijn, Leiden (NL); Jan Pen, Leiden (NL); Josephus Christianus Maria Smeekens, Driebergen (NL); Maria Theresia Smits, Den Haag (NL)

Correspondence Address: HALE AND DORR, LLP, 60 STATE STREET, BOSTON, MA, 02109

(73) Assignee: Mogen International N.V.

(21) Appl. No.: 10/195,962

(22) Filed: Jul. 15, 2002

Related U.S. Application Data
(62) Division of application No. 09/403,837, filed on Mar. 2, 2000, now abandoned.
Application No. 09/403,837 is a 371 of international application No. PCT/EP98/02788, filed on May 4, 1998.

(30) Foreign Application Priority Data
May 2, 1997 (WO) PCT/EP97/02497

(51) Int. Cl. A01H 5/00

(52) U.S. Cl. 800/284; 800/317.2

(57) ABSTRACT

A method of modification of the development and/or composition of cells, tissues, or organs in vivo in plants by inhibiting the level of an endogenous trehalase is provided. The cells, tissues, or organs have been genetically altered to comprise a DNA sequence encoding a trehalase inhibitor. The DNA sequence is capable of expressing an RNA that is at least partially complementary to an RNA produced by a DNA sequence encoding the endogenous trehalase. Alternatively, the DNA sequence comprises a DNA sequence which is identical to a DNA sequence encoding the endogenous trehalase. The modification is other than to increase production or accumulation of trehalose.

### FIELD OF THE INVENTION

Glycolysis has been one of the first metabolic processes described in biochemical detail in the literature. Although the general flow of carbohydrates in organisms is known and although all enzymes of the glycolytic pathway(s) are elucidated, the signal which determines the induction of metabolism by stimulating glycolysis has not been unravelled. Several hypotheses, especially based on the situation in yeast have been put forward, but none has been proven beyond doubt.

Influence on the direction of the carbohydrate partitioning does not only influence directly the cellular processes of glycolysis and carbohydrate storage, but it can also be used to influence secondary or derived processes such as cell division, biomass generation and accumulation of storage compounds, thereby determining growth and productivity.

Especially in plants, often the properties of a tissue are directly influenced by the presence of carbohydrates, and the steering of carbohydrate partitioning can give substantial differences.

The growth, development and yield of plants depends on the energy which such plants can derive from CO₂-fixation during photosynthesis.

Photosynthesis primarily takes place in leaves and to a lesser extent in the stem, while other plant organs such as roots, seeds or tubers do not essentially contribute to the photoassimilation process. These tissues are completely dependent on photosynthetically active organs for their growth and nutrition. This then means that there is a flux of products derived from photosynthesis (collectively called “photosynthate”) to photosynthetically inactive parts of the plants.

The photosynthetically active parts are denominated as “sources” and they are defined as net exporters of photosynthate. The photosynthetically inactive parts are denominated as “sinks” and they are defined as net importers of photosynthate.

It is assumed that both the efficiency of photosynthesis, as well as the carbohydrate partitioning in a plant are essential. Newly developing tissues like young leaves or other parts like root and seed are completely dependent on photosynthesis in the sources. The possibility of influencing the carbohydrate partitioning would have great impact on the phenotype of a plant, e.g. its height, the internodium distance, the size and form of a leaf and the size and structure of the root system.

Furthermore, the distribution of the photoassimilation products is of great importance for the yield of plant biomass and products. An example is the development in wheat over the last century. Its photosynthetic capacity has not changed considerably but the yield of wheat grain has increased substantially, i.e. the harvest index (ratio harvestable biomass/total biomass) has increased. The underlying reason is that the sink-to-source ratio was changed by conventional breeding, such that the harvestable sinks, i.e. seeds, portion increased. However, the mechanism which regulates the distribution of assimilation products and consequently the formation of sinks and sources is yet unknown. The mechanism is believed to be located somewhere in the carbohydrate metabolic pathways and their regulation. In the recent research it has become apparent that hexokinases may play a major role in metabolite signalling and control of metabolic flow. A number of mechanisms for the regulation of the hexokinase activity have been postulated (Graham et al. (1994), The Plant Cell 6: 761; Jang & Sheen (1994). The Plant Cell 6, 1665; Rose et al. Eur. J. Biochem. 199, 511-518, 1991; Blazquez et al. (1993), FEBS 329, 51; Koch, Annu. Rev. Plant Physiol. Plant. Mol. Biol. (1996) 47, 509; Jang et al. (1997), The Plant Cell 9, 5). One of these theories of hexokinase regulation, postulated in yeast, mentions trehalose and its related monosaccharides (Thevelein & Hohmann (1995), TIBS 20, 3). However, it is hard to see that this would be an universal mechanism, as trehalose synthesis is believed to be restricted to certain species. WO 97/42326 shows that trehalose phosphate synthase and trehalose phosphate phosphatase, which both are in the trehalose synthesizing pathway, can induce metabolic changes when transformed to plants. It has been shown in that application that the intracellular level of trehalose-6-phosphate is believed to be the pivotal point.

There still remains a need for other mechanisms which can influence the trehalose-6-phosphate and which thereby can direct the modification of the development and/or composition of cells, tissue and organs in vivo.

### SUMMARY OF THE INVENTION

The invention is directed to a method of modification of the development and/or composition of cells, tissue or organs in vivo by inhibiting endogenous trehalase levels. Part of these are a method for the inhibition of carbon flow in the glycolytic direction in a cell by inhibiting endogenous trehalase levels, a method for the stimulation of photosynthesis by inhibiting endogenous trehalase levels, a method for the stimulation of sink-related activity by inhibiting endogenous trehalase levels, a method for the inhibition of growth of a cell or a tissue by inhibiting endogenous trehalase levels, a method for the prevention of cold sweetening by inhibiting endogenous trehalase levels, a method for the inhibition of invertase in beet after harvest by inhibiting endogenous trehalase levels, a method for the induction of bolting by inhibiting endogenous trehalase levels and a method for increasing the yield in plants by inhibiting endogenous trehalase levels. It is envisaged that the effect of the inhibition of endogenous trehalase levels is caused by an increase of intracellular trehalose-6-phosphate levels. Thus, the invention also provides a method for increasing the intracellular availability of trehalose-6-phosphate by inhibiting endogenous trehalase levels.

The inhibition of endogenous trehalase levels is the result of culturing or growing said cells, tissues, organs or plants in the presence of a trehalase inhibitor. This inhibitor can be validamycin A in a form suitable for uptake by said cells, tissues, organs or plants, preferably wherein the concentration of validamycin A is between 100 nM and 10 mM, more preferably between 0.1 and 1 mM, in aqueous solution. Another option is to use the 86 kD protein of the cockroach (Periplaneta americana) in a form suitable for uptake by said cells, tissue, organs or plants as the inhibitor of the endogenous trehalase levels.

Also part of the invention is to provide the cells, tissue, organs or plants with the genetic information for a trehalase inhibitor. This can be done by transformation with the gene encoding the 86 kD protein of the American cockroach (Periplaneta americana). Alternatively, transformation with a DNA sequence which is capable of expressing an RNA that is at least partially complementary to the RNA produced by the gene encoding the endogenous trehalase or transformation with a DNA sequence coding for the enzyme trehalase which is identical to the DNA sequence encoding the endogenous trehalase.

Specifically the DNA sequence encoding the endogenous trehalase is selected from the group consisting of the nucleotide sequences comprising the nucleotide sequence encoding the protein of SEQ ID NO: 4, the nucleotide sequence encoding the protein of SEQ ID NO: 6, the nucleotide sequence encoding the protein of SEQ ID NO: 8 and the nucleotide sequence encoding the protein of SEQ ID NO: 10, more specifically the DNA sequence encoding the endogenous trehalase is selected from the group consisting of the nucleotide sequences comprising the nucleotide sequence depicted in SEQ ID NO: 3, the nucleotide sequence depicted in SEQ ID NO: 5, the nucleotide sequence depicted in SEQ ID NO: 7 and the nucleotide sequence depicted in SEQ ID NO: 9.

Definitions

Hexokinase activity is the enzymatic activity found in cells which catalyzes the reaction of hexose to hexose-6-phosphate. Hexoses include glucose, fructose, galactose or any other C6 sugar. It is acknowledged that there are many isoenzymes which all can play a part in said biochemical reaction. By catalyzing this reaction hexokinase forms a key enzyme in hexose (glucose) signalling.

Hexose signalling is the regulatory mechanism by which a cell senses the availability of hexose (glucose).

Glycolysis is the sequence of reactions that converts glucose into pyruvate with the concomitant production of ATP.

Storage of resource material is the process in which the primary product glucose is metabolized into the molecular form which is fit for storage in the cell or in a specialized tissue. These forms can be divers. In the plant kingdom storage mostly takes place in the form of carbohydrates and polycarbohydrates such as starch, fructan and cellulose, or as the more simple mono- and di-saccharides like fructose, sucrose and maltose; in the form of oils such as arachic or oleic oil and in the form of proteins such as cruciferin, napin and seed storage proteins in rapeseed. In animal cells also polymeric carbohydrates such as glycogen are formed, but also a large amount of energy rich carbon compounds is transferred into fat and lipids.

Biomass is the total mass of biological material.

### DESCRIPTION OF THE FIGURES

FIG. 1. Schematic representation of plasmid pVDH275 harbouring the neomycin-phosphotransferase gene (NPTII) flanked by the 35S cauliflower mosaic virus promoter (P35S) and terminator (T35S) as a selectable marker; an expression cassette comprising the pea plastocyanin promoter (pPCpea) and the nopaline synthase terminator (Tnos); right (RB) and left (LB) T-DNA border sequences and a bacterial kanamycin resistance (KanR) marker gene.

FIG. 2. Trehalose accumulation in tubers of pMOG1027 (35S as-trehalase) transgenic potato plants.

FIG. 3. Tuber yield of 22 independent wild-type S. tuberosum clones.

FIG. 4. Tuber yield of pMOG1027 (35S as-trehalase) and pMOG1027(845-11/22/28) (35S as-trehalase pat TPS) transgenic potato lines in comparison to wild-type potato lines.

FIG. 5. Starch content of pMOG1027 (35S as-trehalase) and pMOG1027(845-11/22/28) (35S as-trehalase pat TPS) transgenic potato lines in comparison to wild-type potato lines. The sequence of all lines depicted is identical to FIG. 4.

FIG. 6. Yield of pMOG1028 (pat as-trehalase) and pMOG1028(845-11/22/28) (pat as-trehalase pat TPS) transgenic potato lines in comparison to wild-type potato lines.

FIG. 7. Yield of pMOG1092 (PC as-trehalase) transgenic potato lines in comparison to wild-type potato lines as depicted in FIG. 6.

FIG. 8. Yield of pMOG1130 (PC as-trehalase PC TPS) transgenic potato lines in comparison to wild-type potato lines as depicted in FIG. 6.

### DETAILED DESCRIPTION OF THE INVENTION

It has now been found that modification of the development and/or composition of cells, tissue and organs in vivo is possible by inhibiting endogenous trehalase levels thereby inducing a change in the synthetic pathway leading to the formation of trehalose. The inhibition of the endogenous trehalase levels is preferably accomplished by transforming cells with a DNA construct which yields mRNA which is anti-sense to the endogenous trehalase mRNA. Inhibition of trehalase causes inhibition of carbon flow in the glycolytic direction, stimulation of the photosynthesis, stimulation of sink-related activity and an increase in storage of resources.

The invention also gives the ability to modify source-sink relations and resource allocation in plants. The whole carbon economy of the plant, including assimilate production in source tissues and utilization in source tissues can be modified, which may lead to increased biomass yield of harvested products. Using this approach, increased yield potential can be realized, as well as improved harvest index and product quality. These changes in source tissues can lead to changes in sink tissues by for instance increased export of photosynthate. Conversely changes in sink tissue can lead to change in source tissue.

Specific expression in a cell organelle, a tissue or other part of an organism enables the general effects that have been mentioned above to be directed to specific local applications. This specific expression can be established by placing the antisense gene for trehalase under control of a specific promoter.

By using specific promoters it is also possible to construct a temporal difference. For this purpose promoters can be used that are specifically active during a certain period of the organogenesis of the plant parts. In this way it is possible to first influence the amount of organs which will be developed and then enable these organs to be filled with storage material like starch, oil or proteins.

Alternatively, inducible promoters may be used to selectively switch on or off the expression of the genes of the invention. Induction can be achieved by for instance pathogens, stress, chemicals or light/dark stimuli.

The invention is concerned with the finding that metabolism can be modified in vivo by inhibiting endogenous trehalase levels.

These modifications are most likely established by a change in T-6-P levels that in turn affect the signalling function of hexokinase. An increase in the flux through hexokinase (i.e. an increase in the amount of glucose) that is reacted in glucose-6-phosphate has been shown to inhibit photosynthetic activity in plants. Furthermore, an increase in the flux through hexokinase would not only stimulate the glycolysis, but also cell division activity.

Theory of Trehalose-6-Posphate Regulation of Carbon Metabolism

In a normal plant cell formation of carbohydrates takes place in the process of photosynthesis in which CO₂ is fixed and reduced to phosphorylated hexoses with sucrose as an end-product. Normally this sucrose is transported out of the cell to cells or tissues which through uptake of this sucrose can use the carbohydrates as building material for their metabolism or are able to store the carbohydrates as e.g. starch. In this respect, in plants, cells that are able to photosynthesize and thus to produce carbohydrates are denominated as sources, while cells which consume or store the carbohydrates are called sinks.

In animal and most microbial cells no photosynthesis takes place and the carbohydrates have to be obtained from external sources, either by direct uptake from saccharides (e.g. yeasts and other micro-organisms) or by digestion of carbohydrates (animals). Carbohydrate transport usually takes place in these organisms in the form of glucose, which is actively transported over the cell membrane.

After entrance into the cell, one of the first steps in the metabolic pathway is the phosphorylation of glucose into glucose-6-phosphate catalyzed by the enzyme hexokinase. It has been demonstrated that in plants sugars which are phosphorylated by hexokinase (HXK) are controlling the expression of genes involved in photosynthesis (Jang & Sheen (1994), The Plant Cell 6, 1665). Therefore, it has been proposed that HXK may have a dual function and may act as a key sensor and signal transmitter of carbohydrate-mediated regulation of gene-expression. It is believed that this regulation normally signals the cell about the availability of starting product, i.e. glucose. Similar effects are observed by the introduction of TPS or TPP which influence the level of T-6-P. Moreover, it is shown that in vitro, T-6-P levels affect hexokinase activity. By increasing the level of T-6-P, the cell perceives a signal that there is a shortage of carbohydrate input. Conversely, a decrease in the level of T-6-P results in a signal that there is plenty of glucose, resulting in the down-regulation of photosynthesis: it signals that substrate for glycolysis and consequently energy supply for processes as cell growth and cell division is sufficiently available. This signalling is thought to be initiated by the increased flux through hexokinase (J. J. Van Oosten, public lecture at RijksUniversiteit Utrecht dated Apr. 19, 1996).

The theory that hexokinase signalling in plants can be regulated through modulation of the level of trehalose-6-phosphate would imply that all plants require the presence of an enzyme system able to generate and break-down the signal molecule trehalose-6-phosphate. Although trehalose is commonly found in a wide variety of fungi, bacterial, yeasts and algae, as well as in some invertebrates, only a very limited range of vascular plants have been proposed to be able to synthesize this sugar (Elbein (1974), Adv. Carboh. Chem. Biochem. 30, 227). A phenomenon which was not understood until now is that despite the apparent lack of trehalose synthesizing enzymes, all plants do seem to contain trehalases, enzymes which are able to break down trehalose into two glucose molecules.

Indirect evidence for the presence of a metabolic pathway for trehalose is obtained by experiments presented herein with trehalase inhibitors such as Validamycin A or transformation with anti-sense trehalase.

These data indicate that, in contrast to current beliefs, most plants do contain genes which encode trehalose-phosphate-synthases enabling them to synthesize T-6-P. As proven by the accumulation of trehalose in TPS expressing plants, plants also contain phosphatases, non-specific or specific, able to dephosphorylate the T-6-P into trehalose. The presence of trehalase in all plants may be to effectuate turnover of trehalose.

In yeast, a major role of glucose-induced signalling is to switch metabolism from a neogenetic/respirative mode to a fermentative mode. Several signalling pathways are involved in this phenomenon (Thevelein and Hohmann, (1995) TIBS 20, 3). Besides the possible role of hexokinase signalling, the RAS-cyclic-AMP (cAMP) pathway has been shown to be activated by glucose. Activation of the RAS-cAMP pathway by glucose requires glucose phosphorylation, but no further glucose metabolism. So far, this pathway has been shown to activate trehalase and 6-phosphofructo-2-kinase (thereby stimulating glycolysis), while fructose-1,6-bisphosphatase is inhibited (thereby preventing gluconeogenesis), by cAMP-dependent protein phosphorylation. This signal transduction route and the metabolic effects it can bring about can thus be envisaged as one that acts in parallels with the hexokinase signalling pathway, that is shown to be influenced by the level of trehalose-6-phosphate.

In plants, generation of the “plenty” signal by decreasing the intracellular concentration of trehalose-6-phosphate through expression of the enzyme TPP (or inhibition of the enzyme TPS) will signal all cell systems to increase glycolytic carbon flow and inhibit photosynthesis. This is nicely shown in WO 97/42326, where for instance in Experiment 2 transgenic tobacco plants are described in which the enzyme TPP is expressed having increased leaf size, increased branching and a reduction of the amount of chlorophyll. However, since the “plenty” signal is generated in the absence of sufficient supply of glucose, the pool of carbohydrates in the cell is rapidly depleted.

Thus, assuming that the artificial “plenty” signal holds on, the reduction in carbohydrates will finally become limiting for growth and cell division, i.e. the cells will use up all their storage carbohydrates and will be in a “hunger”-stage. Thus, leaves are formed with a low amount of stored carbohydrates. On the other hand, plants that express a construct with a gene coding for TPS, which increases the intracellular amount of T-6-P, showed a reduction of leaf size, while also the leaves were darker green, and contained an increased amount of chlorophyll.

As described in our invention, transgenic plants expressing as-trehalase reveal similar phenomena, like dark-green leaves, enhanced yield, as observed when expressing a TPS gene. Inhibiting endogenous trehalase levels will stop the degradation of trehalose and as a result of the increase in trehalose concentration the enzyme TPP may be inhibited, resulting in increased T-6-P levels. This would explain why inhibition of trehalase has effects similar to the overexpression of TPS. It also seems-that expression of as-trehalase in double-constructs enhances the effects that are caused by the expression of TPS. Trehalase activity has been shown to be present in e.g. plants, insects, animals, fungi and bacteria while only in a limited number of species, trehalose is accumulated.

Up to now, the role of trehalase in plants is unknown although this enzyme is present in almost all plant-species. It has been proposed to be involved in plant pathogen interactions and/or plant defense responses. We have isolated a potato trehalase gene and show that inhibition of trehalase activity in potato leaf and tuber tissues leads to an increase in tuber-yield. Fruit-specific expression of as-trehalase in tomato combined with TPS expression dramatically alters fruit development.

Inhibition of trehalases can be performed basically in two ways: by administration of trehalase inhibitors exogenously, and by the production of trehalase inhibitors endogenously, for instance by transforming the plants with DNA sequences coding for trehalase inhibitors.

According to this first embodiment of the invention, trehalase inhibitors are administered to the plant system exogenously. Examples of trehalase inhibitors that may be used in such a process according to the invention are trehazolin produced in Micromonospora, strain SANK 62390 (Ando et al., 1991, J. Antibiot. 44, 1165-1168), validoxylamine A, B, G, D-gluco-Dihydrovalidoxylamine A, L-ido-Dihydrovalidoxylamin A, Deoxynojirimycin (Kameda et al., 1987, J. Antibiot. 40(4), 563-565), 5epi-trehazolin (Trehalostatin) (Kobayashi Y. et al., 1994, J. Antiobiot. 47, 932-938), castanospermin (Salleh H. M. & Honek J. F. March 1990, FEBS 262(2), 359-362) and the 86kD protein from the american cockroach (Periplaneta americana) (Hayakawa et al., 1989, J. Biol. Chem. 264(27), 16165-16169).

A preferred trehalase inhibitor according to the invention is validamycin A (1,5,6-trideoxy-3-o-β-D-glucopyranosyl-5-(hydroxymethyl)-1-[[4,5,6-trihydroxy-3-(hydroxymenthyl)-2-cyclohexen-1-yl]amino]-D-chiro-inositol). Inhibition of trehalase activity in homogenates of callus and suspension culture of various Angiospermae using Validamycin is disclosed by Kendall et al., 1990, Phytochemistry 29, 2525-2582.

Trehalase inhibitors are administered to plants or plant parts, or plant cell cultures, in a form suitable for uptake by the plants, plant parts or cultures. Typically the trehalase inhibitor is in the form of an aqueous solution of between 100 nM and 10 mM of active ingredient, preferably between 0.1 and 1 mM. Aqueous solutions may be applied to plants or plant parts by spraying on leaves, watering, adding it to the medium of a hydroculture, and the like. Another suitable formulation of validamycin is solacol, a commercially available agricultural formulation (Takeda Chem. Indust., Tokyo).

Alternatively, or in addition to using exogenously administered trehalase inhibitors, trehalase inhibitors may be provided by introducing the genetic information coding therefor. One form of such in-built trehalase inhibitor may consist of a genetic construct causing the production of RNA that is sufficiently complementary to endogenous RNA encoding for trehalase to interact with said endogenous transcript, thereby inhibiting the expression of said transcript. This so-called “antisense approach” is well known in the art (vide inter alia EP 0 240 208 A and the Examples to inhibit SPS disclosed in WO 95/01446). It is preferred to use homologous antisense genes as these are more efficient than heterologous genes. An alternative method to block the synthesis of undesired enzymatic activities is the introduction into the genome of the plant host of an additional copy of an endogenous gene present in the plant host. It is often observed that such an additional copy of a gene silences the endogenous gene: this effect is referred to in the literature as the co-suppressive effect, or co-suppression. Details of the procedure of enhancing substrate availability are provided in the Examples of WO 95/01446, incorporated by reference herein.

Yet another method to inhibit the endogenous trehalase levels is by mutating the endogenous gene coding for trehalase. Effective mutation can be achieved by by introducing mutated gene sequences by site specific mutagenesis (e.g. as described in WO 91/02070).

According to another embodiment of the invention, especially plants can be genetically altered to produce and accumulate the above-mentioned anti-sense gene in specific parts of the plant. Preferred sites of expression are leaves and storage parts of plants. In particular potato tubers are considered to be suitable plant parts. A preferred promoter to achieve selective expression in microtubers and tubers of potato is obtainable from the region upstream of the open reading frame of the patatin gene of potato.

Another suitable promoter for specific expression is the plastocyanin promoter, which is specific for photoassimilating parts of plants. Furthermore, it is envisaged that specific expression in plant parts can yield a favourable effect for plant growth and reproduction or for economic use of said plants. Examples of promoters which are useful in this respect are: the E8-promoter (EP 0 409 629) and the 2A11-promoter (van Haaren and Houck (1993), Plant Mol. Biol., 221, 625) which are fruit-specific; the cruciferin promoter, the napin promoter and the ACP promoter which are seed-specific; the PAL-promoter; the chalcon-isomerase promoter which is flower-specific; the SSU promoter, and ferredoxin promoter, which are leaf-specific; the TobRb7 promoter which is root-specific, the RolC promoter which is specific for phloem and the HMG2 promoter (Enjuto et al. (1995), Plant Cell 7, 517) and the rice PCNA promoter (Kosugi et al. (1995), Plant J. 7, 877) which are specific for meristematic tissue.

Another option under this invention is to use inducible promoters. Promoters are known which are inducible by pathogens, by stress, by chemical or light/dark stimuli. It is envisaged that for induction of specific phenoma, for instance sprouting, bolting, seed setting, filling of storage tissues, it is beneficial to induce the activity of the genes of the invention by external stimuli. This enables normal development of the plant and the advantages of the inducibility of the desired phenomena at control. Promoters which qualify for use in such a regime are the pathogen inducible promoters described in DE 4446342 (fungus and auxin inducible PRP-1), WO 96/28561 (fungus inducible PRP-1), EP 0 586 612 (nematode inducible), EP 0 712 273 (nematode inducible), WO 96/34949 (fungus inducible), PCT/EP96/02437 (nematode inducible), EP 0 330 479 (stress inducible), U.S. Pat. No. 5,510,474 (stress inducible), WO 96/12814 (cold inducible), EP 0 494 724 (tetracycline inducible), EP 0 619 844 (ethylene inducible), EP 0 337 532 (salicylic acid inducible), WO 95/24491 (thiamine inducible) and WO 92/19724 (light inducible). Other chemical inducible promoters are described in EP 0 674 608, EP 637 339, EP 455 667 and U.S. Pat. No. 5,364,780.

Host cells can be any cells in which the modification of hexokinase-signalling can be achieved through alterations in the level of T-6-P. Thus, accordingly, all eukaryotic cells are subject to this invention. From an economic point of view the cells most suited for production of metabolic compounds are most suitable for the invention. These organisms are, amongst others, plants, animals, yeast, fungi. However, also expression in specialized animal cells (like pancreatic beta-cells and fat cells) is envisaged.

Preferred plant hosts among the Spermatophytae are the Angiospermae, notably the Dicotyledoneae, comprising inter alia the Solanaceae as a representative family, and the Monocotyledoneae, comprising inter alia the Gramineae as a representative family. Suitable host plants, as defined in the context of the present invention include plants (as well as parts and cells of said plants) and their progeny which contain a modified level of T-6-P by inhibition of the endogenous trehalase levels. Crops according to the invention include those which have flowers such as cauliflower (Brassica oleracea), artichoke (Cynara scolymus), cut flowers like carnation (Dianthus caryophyllus), rose (Rosa spp), Chrysanthemum, Petunia, Alstromeria, Gerbera, Gladiolus, lily (Lilium spp), hop (Humulus lupulus), broccoli, potted plants like Rhododendron, Azalia, Dahlia, Begonia, Fuchsia, Geranium etc.; fruits such as apple (Malus, e.g. domesticus), banana (Musa, e.g. Acuminata), apricot (Prunus armeniaca), olive (Oliva sativa), pineapple (Ananas comosus), coconut (Cocos nucifera), mango (Mangifera indica), kiwi, avocado (Persea americana), berries (such as the currant, Ribes, e.g. rubrum), cherries (such as the sweet cherry, Prunus, e.g. avium), cucumber (Cucumis, e.g. sativus), grape (Vitis, e.g. vinifera), lemon (Citrus limon), melon (Cucumis melo), mustard (Sinapis alba and Brassica nigra), nuts (such as the walnut, Juglans, e.g. regia; peanut, Arachis hypogeae), orange (Citrus, e.g. maxima), peach (Prunus, e.g. persica), pear (Pyra, e.g. Communis), pepper (Solanum, e.g. capsicum), plum (Prunus, e.g. domestica), strawberry (Fragaria, e.g. moschata), tomato (Lycopersicon, e.g. esculentum); leaves, such as alfalfa (Medicago sativa), cabbages (such as Brassica oleracea), endive (Cichoreum, e.g. endivia), leek (Allium porrum), lettuce (Lactuca sativa), spinach (Spinacia oleraceae), tobacco (Nicotiana tabacum), grasses like Festuca, Poa, rye-grass (such as Lolium perenne, Lolium multiflorum and Arrenatherum spp.), amenity grass, turf, seaweed, chicory (Cichorium intybus), tea (Thea sinensis), celery, parsley (Petroselinum crispum), chevil and other herbs; roots, such as arrowroot (Maranta arundinacea), beet (Beta vulgaris), carrot (Daucus carota), cassava (Manihot esculenta), ginseng (Panax ginseng), turnip (Brassica rapa), radish (Raphanus sativus), yam (Dioscorea esculenta), sweet potato (Ipomoea batatas), taro; seeds, such as beans (Phaseolus vulgaris), pea (Pisum sativum), soybean (Glycin max), wheat (Triticum aestivum), barley (Hordeum vulgare), corn (Zea mays), rice (Oryza sativa), bush beans and broad beans (Vicia faba), cotton (Gossypium spp.), coffee (Coffea arabica and C. canephora); tubers, such as kohlrabi (Brassica oleraceae), potato (Solanum tuberosum); bulbous plants as onion (Allium cepa), scallion, tulip (Tulipa spp.), daffodil (Narcissus spp.), garlic (Allium sativum); stems such as cork-oak. sugarcane (Saccharum spp.) , sisal (Sisal spp.) flax (Linum vulgare), jute; trees like rubber tree, oak (Quercus spp.) , beech (Betula spp.), alder (Alnus spp.), ashtree (Acer spp.), elm (Ulmus spp.), palms, ferns, ivies and the like.

Transformation of yeast and fungal or animal cells can be done through normal state-of-the art transformation techniques through commonly known vector systems like pBluescript, pUC and viral vector systems like RSV and SV40.

The method of introducing the genes into a recipient plant cell is not crucial, as long as the gene is expressed in said plant cell.

Although some of the embodiments of the invention may not be practicable at present, e.g. because some plant species are as yet recalcitrant to genetic transformation, the practicing of the invention in such plant species is merely a matter of time and not a matter of principle, because the amenability to genetic transformation as such is of no relevance to the underlying embodiment of the invention.

Transformation of plant species is now routine for an impressive number of plant species, including both the Dicotyledoneae as well as the Monocotyledoneae. In principle any transformation method may be used to introduce chimeric DNA according to the invention into a suitable ancestor cell. Methods may suitably be selected from the calcium/polyethylene glycol method for protoplasts (Krens et al. (1982), Nature 296, 72; Negrutiu et al. (1987), Plant Mol. Biol. 8, 363, electroporation of protoplasts (Shillito et al. (1985) Bio/Technol. 3, 1099), microinjection into plant material (Crossway et al. (1986), Mol. Gen. Genet. 202), (DNA or RNA-coated) particle bombardment of various plant material (Klein et al. (1987), Nature 327, 70), infection with (non-integrative) viruses, in planta Agrobacterium tumefaciens mediated gene transfer by infiltration of adult plants or transformation of mature pollen or microspores (EP 0 301 316) and the like. A preferred method according to the invention comprises Agrobacterium-mediated DNA transfer. Especially preferred is the use of the so-called binary vector technology as disclosed in EP A 120 516 and U.S. Pat. No. 4,940,838).

Although considered somewhat more recalcitrant towards genetic transformation, monocotyledonous plants are amenable to transformation and fertile transgenic plants can be regenerated from transformed cells or embryos, or other plant material. Presently, preferred methods for transformation of monocots are microprojectile bombardment of embryos, explants or suspension cells, and direct DNA uptake or (tissue) electroporation (Shimamoto et al. (1989), Nature 338, 274-276). Transgenic maize plants have been obtained by introducing the Streptomyces hygroscopicus bar-gene, which encodes phosphinothricin acetyltransferase (an enzyme which inactivates the herbicide phosphinothricin), into embryogenic cells of a maize suspension culture by microprojectile bombardment (Gordon-Kamm (1990), Plant Cell, 2, 603). The introduction of genetic material into aleurone protoplasts of other monocot crops such as wheat and barley has been reported (Lee (1989), Plant Mol. Biol. 13, 21). Wheat plants have been regenerated from embryogenic suspension culture by selecting embryogenic callus for the establishment of the embryogenic suspension cultures (Vasil (1990) Bio/Technol. 8, 429). The combination with transformation systems for these crops enables the application of the present invention to monocots.

Monocotyledonous plants, including commercially important crops such as rice and corn are also amenable to DNA transfer by Agrobacterium strains (vide WO 94/00977; EP 0 159 418 B1; Gould et al. (1991) Plant. Physiol. 95, 426-434).

It is known that practically all plants can be regenerated from cultured cells or tissues. The means for regeneration vary from species to species of plants, but generally a suspension of transformed protoplasts or a petri plate containing transformed explants is first provided. Shoots may be induced directly, or indirectly from callus via organogenesis or embryogenesis and subsequently rooted. Next to the selectable marker, the culture media will generally contain various amino acids and hormones, such as auxin and cytokinins. It is also advantageous to add glutamic acid and proline to the medium, especially for such species as corn and alfalfa. Efficient regeneration will depend on the medium, on the genotype and on the history of the culture. If these three variables are controlled regeneration is usually reproducible and repeatable. After stable incorporation of the transformed gene sequences into the transgenic plants, the traits conferred by them can be transferred to other plants by sexual crossing. Any of a number of standard breeding techniques can be used, depending upon the species to be crossed.

Suitable DNA sequences for control of expression of the plant expressible genes (including marker genes), such as transcriptional initiation regions, enhancers, non-transcribed leaders and the like, may be derived from any gene that is expressed in a plant cell. Also intended are hybrid promoters combining functional portions of various promoters, or synthetic equivalents thereof. Apart from constitutive promoters, inducible promoters, or promoters otherwise regulated in their expression pattern, e.g. developmentally or cell-type specific, may be used to control expression of the expressible genes according to the invention.

To select or screen for transformed cells, it is preferred to include a marker gene linked to the plant expressible gene according to the invention to be transferred to a plant cell. The choice of a suitable marker gene in plant transformation is well within the scope of the average skilled worker; some examples of routinely used marker genes are the neomycin phosphotransferase genes conferring resistance to kanamycin (EP-B 131 623), the glutathion-S-transferase gene from rat liver conferring resistance to glutathione derived herbicides (EP-A 256 223), glutamine synthetase conferring upon overexpression resistance to glutamine synthetase inhibitors such as phosphinothricin (WO 87/05327), the acetyl transferase gene from Streptomyces viridochromogenes conferring resistance to the selective agent phosphinothricin (EP-A 275 957), the gene encoding a 5-enolshikimate-3-phosphate synthase (EPSPS) conferring tolerance to N-phosphonomethylglycine, the bar gene conferring resistance against Bialaphos (e.g. WO 91/02071), the cah gene conferring resistance to cyanamide and the like. The actual choice of the marker is not crucial as long as it is functional (i.e. selective) in combination with the plant cells of choice.

The marker gene and the gene of interest do not have to be linked, since co-transformation of unlinked genes (U.S. Pat. No. 4,399,216) is also an efficient process in plant transformation.

Preferred plant material for transformation, especially for dicotyledonous crops are leaf-discs which can be readily transformed and have good regenerative capability (Horsch et al. (1985), Science 227, 1229).

In animals or human beings it is envisaged that diseases caused by a defect in metabolism can be overcome by inhibiting endogenous trehalase levels in the affected cells. In human cells, the increased glucose consumption of many tumour cells depends to a large extent on the overexpression of hexokinase (Rempel et al. (1996) FEBS Lett. 385, 233). It is envisaged that the flux of glucose into the metabolism of cancer cells can be influenced by the expression of trehalose-6-phosphate synthesizing enzymes and by the inhibition of the endogenous trehalase. It has also been shown that the hexokinase activation is potentiated by the cAMP/PRA (protein kinase A pathway). Therefore, inactivation of this signal transduction pathway may affect glucose uptake and the proliferation of neoplasias. Enzyme activities in mammalian cells able to synthesize trehalose-6-phosphate and trehalose and degrade trehalose have been shown in e.g. rabbit kidney cortex cells (Sacktor (1968) Proc. Natl.Acad.Sci. USA 60, 1007).

As is already acknowledged above, inhibition of the endogenous trehalase levels, e.g. by the introduction of an anti-sense trehalase construct will also stimulate similar effects as the introduction of TPS. These effects have been found to be an increase in the amount of T-6-P which causes dwarfing or stunted growth (especially at high expression of TPS), formation of more lancet-shaped leaves, darker colour due to an increase in chlorophyll and an increase in starch content. Moreover, the use of double-constructs of TPS and as-trehalase enhances the effects of a single construct.

Increase in the level of T-6-P also causes an increase in the storage carbohydrates such as starch and sucrose. This then would mean that tissues in which carbohydrates are stored would be able to store more material. This can be illustrated by the Examples where it is shown that in plants increased biomass of storage organs such as tubers and thickened roots as in beets (storage of sucrose) are formed.

Crops in which this would be very advantageous are potato, sugarbeet, carrot, chicory and sugarcane.

An additional economically important effect in potatoes is that after transformation with DNA encoding for the TPS gene (generating an increase in T-6-P) it has been found that the amount of soluble sugars decreases, even after harvest and storage of the tubers under cold conditions (4° C.). Normally even colder storage would be necessary to prevent early sprouting, but this results in excessive sweetening of the potatoes. Reduction of the amount of reducing sugars is of major importance for the food industry since sweetened potato tuber material is not suitable for processing because a Maillard reaction will take place between the reducing sugars and the amino-acids which results in browning.

In the same way also inhibition of activity of invertase can be obtained by transforming sugarbeets with a polynucleotide encoding for the enzyme TPS. Inhibition of invertase activity in sugarbeets after harvest is economically very important.

Also in fruits and seeds, storage can be altered. This does not only result in an increased storage capacity but in a change in the composition of the stored compounds. Crops in which improvements in yield in seed are especially important are maize, rice, cereals, pea, oilseed rape, sunflower, soybean and legumes. Furthermore, all fruitbearing plants are important for the application of developing a change in the amount and composition of stored carbohydrates. Especially for fruit the composition of stored products gives changes in solidity and firmness, which is especially important in soft fruits like tomato, banana, strawberry, peach, berries and grapes.

In contrast to the effects seen with the decrease of T-6-P levels, an increase in T-6-P levels reduces the ratio of protein/carbohydrate in leaves. This effect is of importance in leafy crops such as fodder grasses and alfalfa. Furthermore, the leaves have a reduced biomass, which can be of importance in amenity grasses, but, more important, they have a relatively increased energy content. This property is especially beneficial for crops as onion, leek and silage maize.

Furthermore, also the viability of the seeds can be influenced by the level of intracellularly available T-6-P.

Combinations of lower levels of T-6-P in one part of a plant and increased levels of T-6-P in another part of the plant can synergize to increase the above-described effects. It is also possible to express the genes driving said decrease or increase sequential during development by using specific promoters. Lastly, it is also possible to induce expression of either of the genes involved by placing the coding the sequence under control of an inducible promoter. It is envisaged that combinations of the methods of application as described will be apparent to the person skilled in the art.

The invention is further illustrated by the following examples. It is stressed that the Examples show specific embodiments of the inventions, but that it will be clear that variations on these examples and use of other plants or expression systems are covered by the invention.

Experimental

DNA Manipulations

All DNA procedures (DNA isolation from E.coli, restriction, ligation, transformation, etc.) are performed according to standard protocols (Sambrook et al. (1989) Molecular Cloning: a laboratory manual, 2nd ed. Cold Spring Harbor Laboratory Press, CSH, N.Y.).

Strains

In all examples E.coli K-12 strain DH5α is used for cloning. The Agrobacterium tumefaciens strains used for plant transformation experiments are EHA 105 and MOG 101 (Hood et al. (1993) Trans. Research 2, 208).

Construction of Agrobacterium Strain MOG101

Construction of Agrobacterium strain MOG101 is described in WO 96/21030.

Cloning of the E.coli otsA Gene and Construction of pMOG799

In E.coli trehalose phosphate synthase (TPS) is encoded by the otsA gene located in the operon otsBA. The cloning and sequence determination of the otsA gene is described in detail in Example I of WO95/01446, herein incorporated by reference. To effectuate its expression in plant cells, the open reading frame has been linked to the transcriptional regulatory elements of the CaMV 35S RNA promoter, the translational enhancer of the ALMV leader, and the transcriptional terminator of the nos-gene, as described in greater detail in Example I of WO95/01446, resulting in pMOG799. A sample of an E. coli strain harbouring pMOG799 has been deposited under the Budapest Treaty at the Centraal Bureau voor Schimmelcultures, Oosterstraat 1, P.O. Box 273, 3740 AG Baarn, The Netherlands, on Monday Aug. 23, 1993: the Accession Number given by the International Depositary Institution is CBS 430.93.

Isolation of a Patatin Promoter/Construction of pMOG546

A patatin promoter fragment is isolated from chromosomal DNA of Solanum tuberosum cv. Bintje using the polymerase chain reaction. A set of oligonucleotides, complementary to the sequence of the upstream region of the λpat21 patatin gene (Bevan et al. (1986) Nucl. Acids Res. 14, 5564), is synthesized consisting of the following sequences:

(SEQIDNO:1)

| | |
|---|---|
| 5′ AAG CTT ATG TTG CCA TAT AGA GTA G 3′ | PatB33.2 |

(SEQIDNO:2)

| | |
|---|---|
| 5′ GTA GTT GCC ATG GTG CAA ATG TTC 3′ | PatATG.2 |

These primers are used to PCR amplify a DNA fragment of 1123bp, using chromosomal DNA isolated from potato cv. Bintje as a template. The amplified fragment shows a high degree of similarity to the λpat21 patatin sequence and is cloned using EcoRI linkers into a pUC18 vector resulting in plasmid pMOG546.

Construction of pMOG845

Construction of pMOG845 is described in WO 96/21030.

Construction of pVDH318, Plastocvanin-TPS

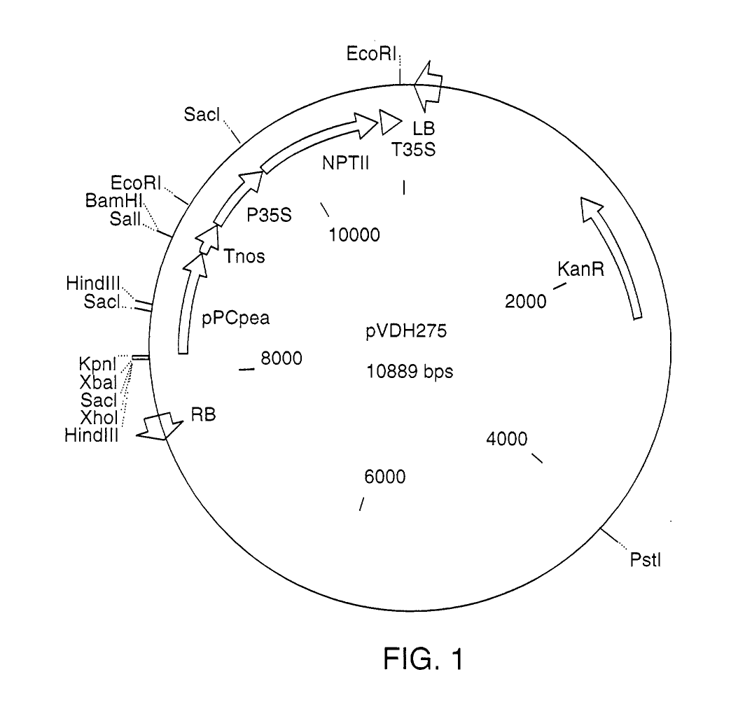

Plasmid pMOG798 (described in WO95/01446) is digested with HindIII and ligated with the oligonucleotide duplex TCV11 and TCV12 (see construction of pMOG845). The resulting vector is digested with PstI and HindIII followed by the insertion of the PotPiII terminator resulting in pTCV118. Plasmid pTCV118 is digested with SmaI and HindIII yielding a DNA fragment comprising the TPS coding region and the PotPiII terminator. BglII linkers were added and the resulting fragment was inserted in the plant binary expression vector pVDH275 (FIG. 1) digested with BamHI, yielding pVDH318. pVDH275 is a derivative of pMOG23 (Sijmons et al. (1990), Bio/Technol. 8. 217) harbouring the NPTII selection marker under control of the 35S CaMV promoter and an expression cassette comprising the pea plastocyanin (PC) promoter and nos terminator sequences. The plastocyanin promoter present in pVDH275 has been described by Pwee & Gray (1993) Plant J. 3, 437. This promoter has been transferred to the binary vector using PCR amplification and primers which contain suitable cloning sites.

Construction of Other Expression Vectors

Similar to the construction of the above mentioned vectors, gene constructs can be made where different promoters are used, in combination with TPS, TPP or trehalase using binary vectors with the NPTII gene or the Hygromycin-resistance gene as selectable marker gene. A description of binary vector pMOG22 harbouring a HPT selection marker is given in Goddijn et al. (1993) Plant J. 4, 863.

Triparental Matings

The binary vectors are mobilized in triparental matings with the E. coli strain HB101 containing plasmid pRK2013 (Ditta et al. (1980) Proc. Natl. Acad. Sci. USA 77, 7347) into Agrobacterium tumefaciens strain MOG101 or EHA105 and used for transformation.

Transformation of Tobacco (Nicotiana tabacum cv. SR1 or cv. Samsun NN)

Tobacco was transformed by cocultivation of plant tissue with Agrobacterium tumefaciens strain MOG101 containing,the binary vector of interest as described. Transformation was carried out using cocultivation of tobacco leaf disks as described by Horsch et al. (1985) Science 227, 1229. Transgenic plants are regenerated from shoots that grow on selection medium containing kanamycin, rooted and transferred to soil.

Transformation of Potato

Potato (Solanum tuberosum cv. Kardal) was transformed with the Agrobacterium strain EHA 105 containing the binary vector of interest. The basic culture medium was MS30R3 medium consisting of MS salts (Murashige and Skoog (1962) Physiol. Plant. 14, 473), R3 vitamins (Ooms et al. (1987) Theor. Appl. Genet. 73, 744), 30 g/l sucrose, 0.5 g/l MES with final pH 5.8 (adjusted with KOH) solidified when necessary with 8 g/l Daichin agar. Tubers of Solanum tuberosum cv. Kardal were peeled and surface sterilized by burning them in 96% ethanol for 5 seconds. The flames were extinguished in sterile water and cut slices of approximately 2 mm thickness. Disks were cut with a bore from the vascular tissue and incubated for 20 minutes in MS30R3 medium containing 1-5×10⁸ bacteria/ml of Agrobacterium EHA 105 containing the binary vector. The tuber discs were washed with MS30R3 medium and transferred to solidified postculture medium (PM). PM consisted of M30R3 medium supplemented with 3.5 mg/l zeatin riboside and 0.03 mg/l indole acetic acid (IAA). After two days, discs were transferred to fresh PM medium with 200 mg/l cefotaxim and 100 mg/l vancomycin. Three days later, the tuber discs were transferred to shoot induction medium (SIM) which consisted of PM medium with 250 mg/l carbenicillin and 100 mg/l kanamycin. After 4-8 weeks, shoots emerging from the discs were excised and placed on rooting medium (MS30R3-medium with 100 mg/l cefotaxim, 50 mg/l vancomycin and 50 mg/l kanamycin). The shoots were propagated axenically by meristem cuttings.

Transformation of Lycopersicon Esculentum

Tomato transformation was performed according to Van Roekel et al. (1993) Plant Cell Rep. 12, 644.

Induction of Micro-tubers

Stem segments of in vitro potato plants harbouring an auxiliary meristem were transferred to micro-tuber inducing medium. Micro-tuber inducing medium contains 1×MS-salts supplemented with R3 vitamins, 0.5 g/l MES (final pH=5.8, adjusted with KOH) and solidified with 8 g/l Daichin agar, 60 g/l sucrose and 2.5 mg/l kinetin. After 3 to 5 weeks of growth in the dark at 24° C., micro-tubers were formed.

Isolation of Validamycin A

Validamycin A has been found to be a highly specific inhibitor of trehalases from various sources ranging from (IC₅₀) 10⁻⁶M to 10⁻¹⁰M (Asano et al. (1987) J. Antibiot. 40. 526; Kameda et al. (1987) J. Antibiot.40, 563). Except for trehalase, it does not significantly inhibit any α- or β-glycohydrolase activity. Validamycin A was isolated from Solacol, a commercial agricultural formulation (Takeda Chem. Indust., Tokyo) as described by Kendall et al. (1990) Phytochemistry 29, 2525. The procedure involves ion-exchange chromatography (QAE-Sephadex A-25 (Pharmacia), bed vol. 10 ml, equilibration buffer 0.2 mM Na-Pi pH 7) from a 3% agricultural formulation of Solacol. Loading 1 ml of Solacol on the column and eluting with water in 7 fractions, practically all Validamycin was recovered in fraction 4. Based on a 100% recovery, using this procedure, the concentration of Validamycin A was adjusted to 1.10⁻³M in MS-medium, for use in trehalose accumulation tests. Alternatively, Validamycin A and B may be purified directly from Streptomyces hygroscopicus var. limoneus, as described by Iwasa et al. (1971) J. Antibiot. 24, 119, the content of which is incorporated herein by reference.

Carbohydrate Analysis

Carbohydrates were determined quantitatively by anion exchange chromatography with pulsed electrochemical detection. Extracts were prepared by extracting homogenized frozen material with 80% EtOH. After extraction for 15 minutes at room temperature, the soluble fraction is evaporated and dissolved in distilled water. Samples (25 μl) were analyzed on a Dionex DX-300 liquid chromatograph equipped with a 4×250 mm Dionex 35391 carbopac PA-1 column and a 4×50 mm Dionex 43096 carbopac PA-1 precolumn. Elution was with 100 mM NaOH at 1 ml/min followed by a NaAc gradient. Sugars were detected with a pulsed electrochemical detector (Dionex, PED). Commercially available carbohydrates (Sigma) were used as a standard.

Determination of Trehalose-6-Phosphate

Leafdiscs (three) of 1.1 cm diameter were frozen in liquid nitrogen and homogenized with 1.5 ml MeOH (80% v/v) using a metal rod. The sample is heated for 15 minutes at 75° C. and dried in a SpeedVac. The pellet was extracted using 450 μl water and stored on ice before injection on the HPLC. The Dionex system as described above was used with the following gradient: T=0′-20′ equilibration with 75 mM NaOH (constant during entire run), T=20′ is time of injection, T=40′-50′ linear increase of 0-10% of 1M NaAc, T=60′-100′ linear increase of 10-50% of 1M NaAc, T=120′ is end of run. The retention times and concentrations of the peaks identified were respectively compared and calculated using a sugar standard solution.

Starch Analysis

Starch analysis was performed as described in: Aman et al. (1994) Methods in Carbohydrate Chemistry, Volume X (eds. BeMiller et al.), pp 111-115.

Expression Analysis

The expression of genes introduced in various plant species was monitored using Northern blot analysis.

### EXAMPLE 1

Inhibition of Trehalase Activity Results in the Accumulation of Trehalose

Transgenic potato plants were generated harbouring the otsA gene driven by the potato tuber-specific patatin promoter (pMOG845). Potato Solanum tuberosum cv. Kardal tuber discs were transformed with Agrobacterium tumefaciens EHA105 harbouring the binary vector pMOG845. Transgenics were obtained with transformation frequencies comparable to empty vector controls. All plants obtained were phenotypically indistinguishable from wild type plants. Micro-tubers were induced on stem segments of transgenic and wild-type plants cultured on microtuber-inducing medium supplemented with 10⁻³M Validamycin A. As a control, microtubers were induced on medium without Validamycin A. Microtubers induced on medium with Validamycin A showed elevated levels of trehalose in comparison with microtubers grown on medium without Validamycin A (table 1) indicating that the trehalase activity present is degrading the formed trehalose. The presence of small amounts of trehalose in wild-type plants indicates the presence of a functional trehalose biosynthetic pathway.

TABLE 1
Trehalose (% fresh weight)

| | +Validamycin A | −Validamycin A |
|---|---|---|
| 845-2 | 0.016 | — |
| 845-4 | — | — |
| 845-8 | 0.051 | — |
| 845-11 | 0.015 | — |
| 845-13 | 0.011 | — |
| 845-22 | 0.112 | — |
| 845-25 | 0.002 | — |
| 845-28 | 0.109 | — |
| wild-type Kardal | 0.001 | — |

### EXAMPLE 2

Trehalose Accumulation in Potato Plants Transgenic for As-trehalase

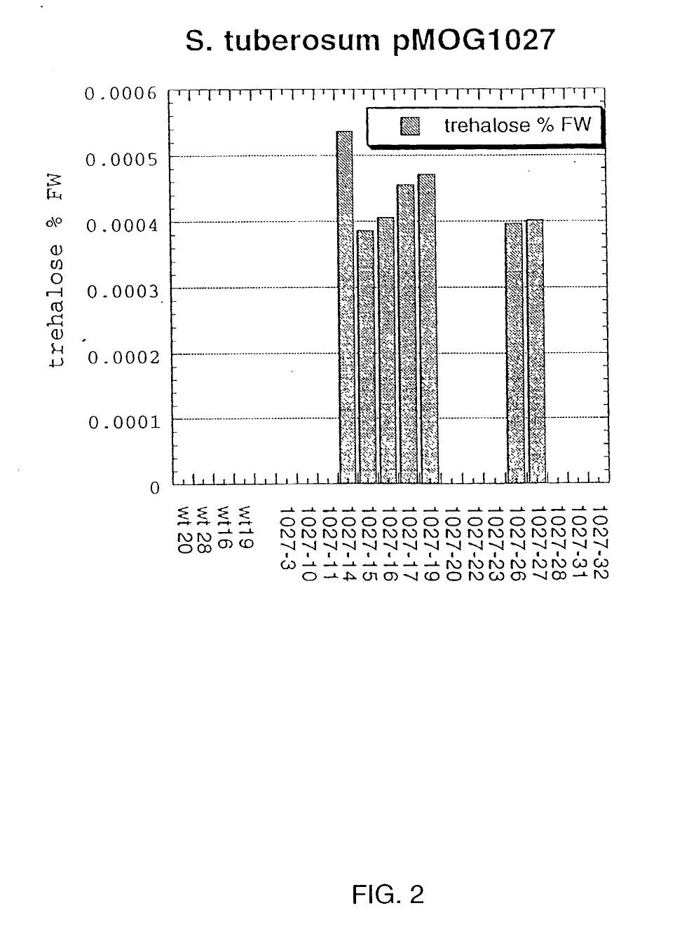

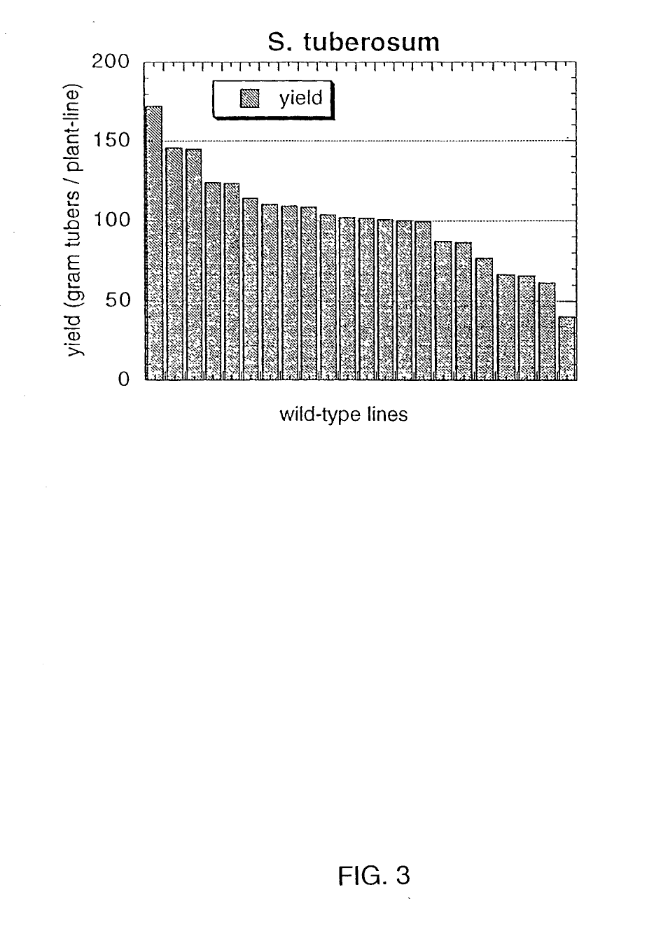

Proof for the presence of an endogenous trehalose biosynthetic pathway was obtained by transforming wild-type potato plants with a 35S CaMV anti-sense trehalase construct (SEQ ID NO: 3 and 4; pMOG1027 is described in WO 96/21030). A potato shoot transgenic for pMOG1027 showed to accumulate trehalose up to 0.008% on a fresh weight basis. The identity of the trehalose peak observed was confirmed by specificly breaking down the accumulated trehalose with the enzyme trehalase. Tubers of some pMOG1027 transgenic lines showed to accumulate small amounts of trehalose (FIG. 2)

TABLE 2

| | Genbank | | |
|---|---|---|---|
| dbEST | Accession | | |
| ID. | No. | Organism | Function |
| 680701 | AA054930 | Brugia malayi | trehalase |
| 693476 | C12818 | Caenorhabditis | trehalase |
| | | elegans | |
| 914068 | AA273090 | Brugia malayi | trehalase |
| 15008 | T00368 | C. elegans | trehalase |
| 401537 | D67729 | C. elegans | trehalase |
| 680728 | AA054884 | Brugia malayi | trehalase |
| 694414 | C13756 | C. elegans | trehalase |
| 871371 | AA231986 | Brugia malayi | trehalase |
| 894468 | AA253544 | Brugia malayi | trehalase |

### EXAMPLE 3

Identification of EST Clones Homologous to the Isolated Potato Trehalase cDNA

The isolation of a potato cDNA clone encoding trehalase was described in WO 96/21030. Comparison of the potato trehalase sequence with EST sequences (expressed sequence tags) indicates the presence of highly homologous genes in various organisms (see Table 2).

### EXAMPLE 4

Isolation of a Tobacco cDNA Clone Encoding Trehalase

In order to be able to study the down-regulation of trehalase expression in tobacco, a tobacco trehalase cDNA was isolated. A cDNA library was constructed in lambda ZAP using the SMART PCR cDNA Construction kit (Clontech). As starting material 1 ug total RNA of wild-type tobacco leaves was used. In total 10⁶ p.f.u. were plated and hybridized with the potato trehalase cDNA. Five positive clones were identified. In vivo excision of one of these clones in ABLE C/K resulted in plasmid pMOG1261, harbouring an insert of ca. 1.3 kb. Nucleic acid sequencing revealed extensive homology to the potato trehalase cDNA sequence confirming the identity of this tobacco trehalase cDNA (SEQ ID NO: 5 and 6, SEQ ID NO: 7 and 8).

### EXAMPLE 5

Performance of Tomato Plants Transgenic for TPS and As-trehalase

Constructs used in tomato transformation experiments: PC-TPS, PC-TPS as-trehalase, E8-TPS, E8 TPS E8 as-trehalase. Plants transgenic for the TPS gene driven by the plastocyanin promoter and 35S promoter did not form small lancet shaped leaves although some severely stunted plants did form small dark-green leaves. Plants transgenic for PC-TPS and PC-as-trehalase did form smaller and darker green leaves as compared to control plants. Similar to what has been observed for TPS and TPP in other crops (WO 97/42326) the colour and leaf-edge of the 35S or PC driven TPS transgenic plants were clearly distinguishable. Only some plants harbouring the TPS gene under control of the fruit-specific E8 promoter did a yellow skin and incomplete ripening. This in contrast to the large number of plants transgenic for E8 TPS E8 as-trehalase producing aberrant fruits with a yellow skin and incomplete ripening.

### EXAMPLE 6

Performance of Potato Plants Transgenic for As-trehalase and/or TPS

Constructs: 35S as-trehalase (pMOG1027) and 35S as-trehalase Pat TPS (pMOG1027(845-11/22/28).

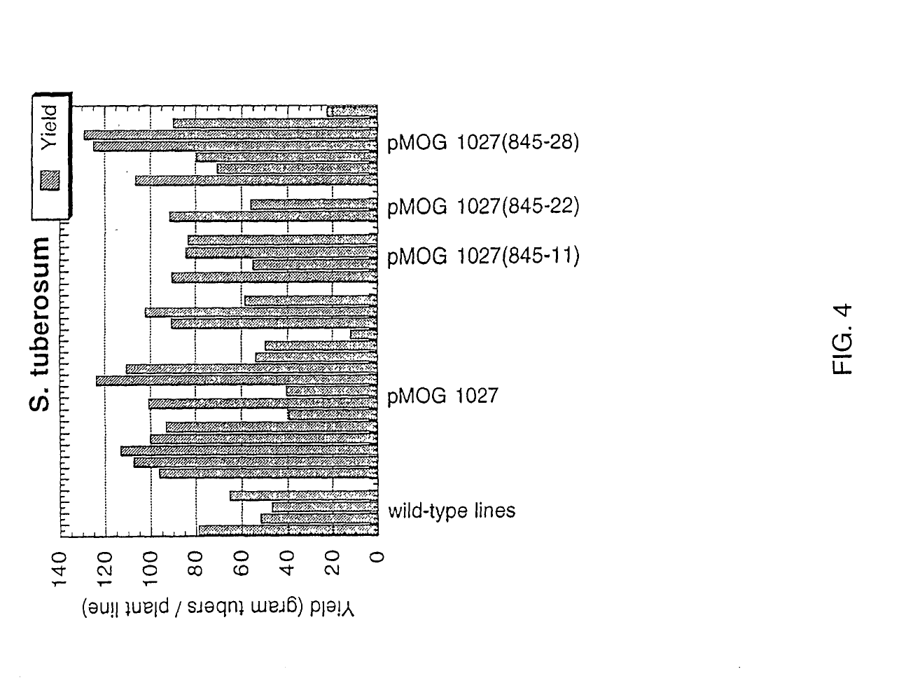

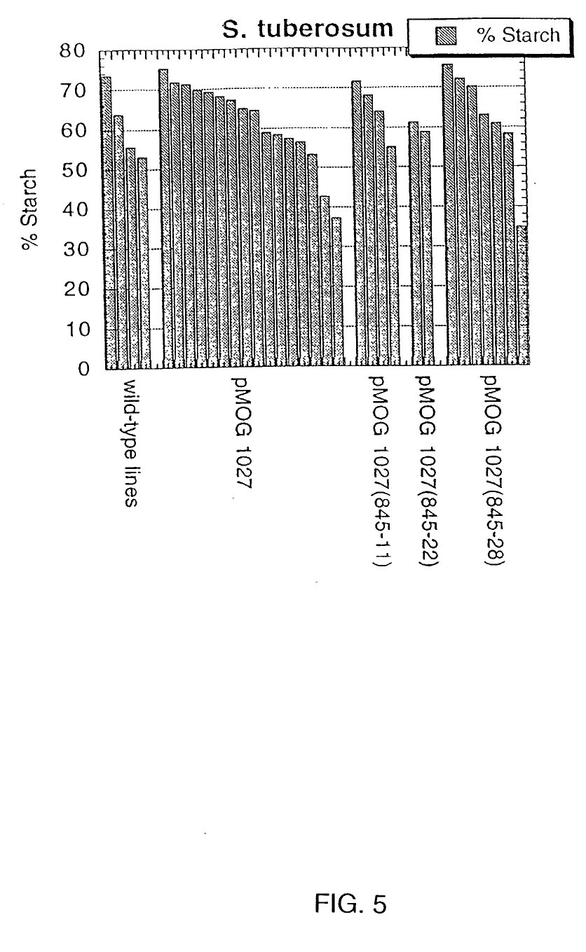

Plants expressing 35S as-trehalase and pat-TPS simultaneously were generated by retransforming pat-TPS lines (resistant against kanamycin) with construct pMOG1027, harbouring the 35S as-trehalase construct and a hygromycin resistance marker gene, resulting in genotypes pMOG1027(845-11, pMOG1027(845-22) and pMOG1027(845-28). Microtubers were induced in vitro and fresh weight of the microtubers was determined. The average fresh weight yield was increased for transgenic lines harbouring pMOG1027 (pMOG845-11/22/28). The fresh weight biomass of microtubers obtained from lines transgenic for pMOG1027 only was slightly higher then wild-type control plants. Resulting plants were grown in the greenhouse and tuber yield was determined (FIG. 4). Lines transgenic for 35S as-trehalase or a combination of 35S as-trehalase and pat-TPS yielded significantly more tuber-mass compared to control lines. Starch determination revealed no difference in starch content of tubers produced by plant lines having a higher yield (FIG. 5). A large number of the 1027(845-11/22/28) lines produced tubers above the soil out of the axillary buds of the leaves indicating a profound influence of the constructs used on plant development. Plant lines transgenic for 35S as-trehalase only did not form tubers above the soil.

Constructs: Pat as-trehalase (pMOG1028) and Pat as-trehalase Pat TPS (pMOG1028(845-11/22/28))

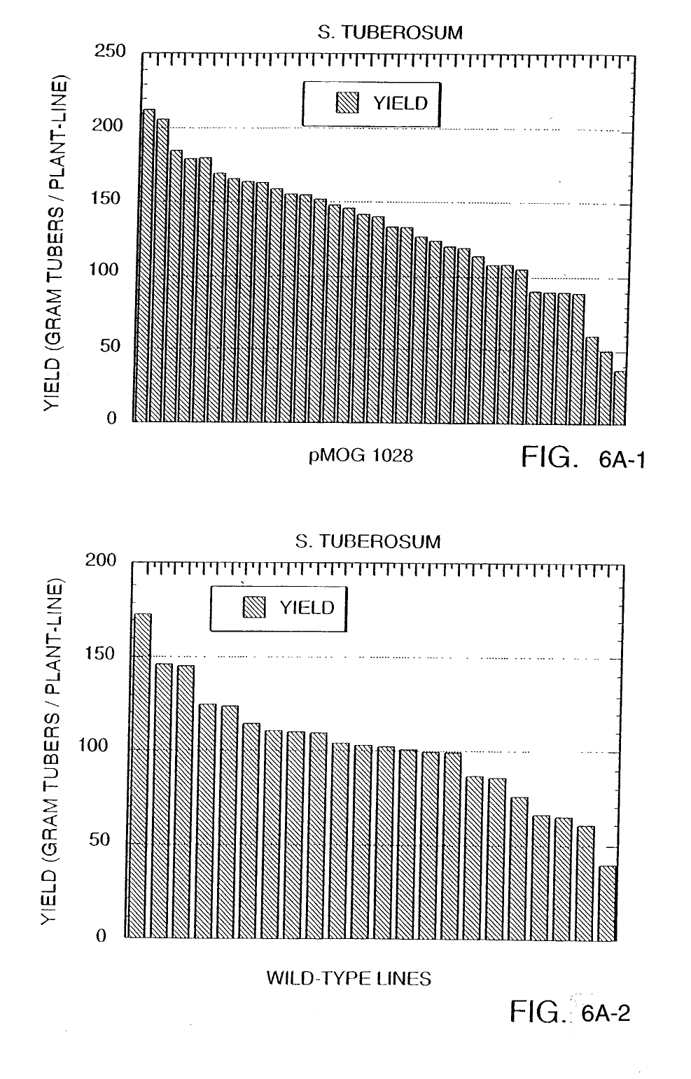

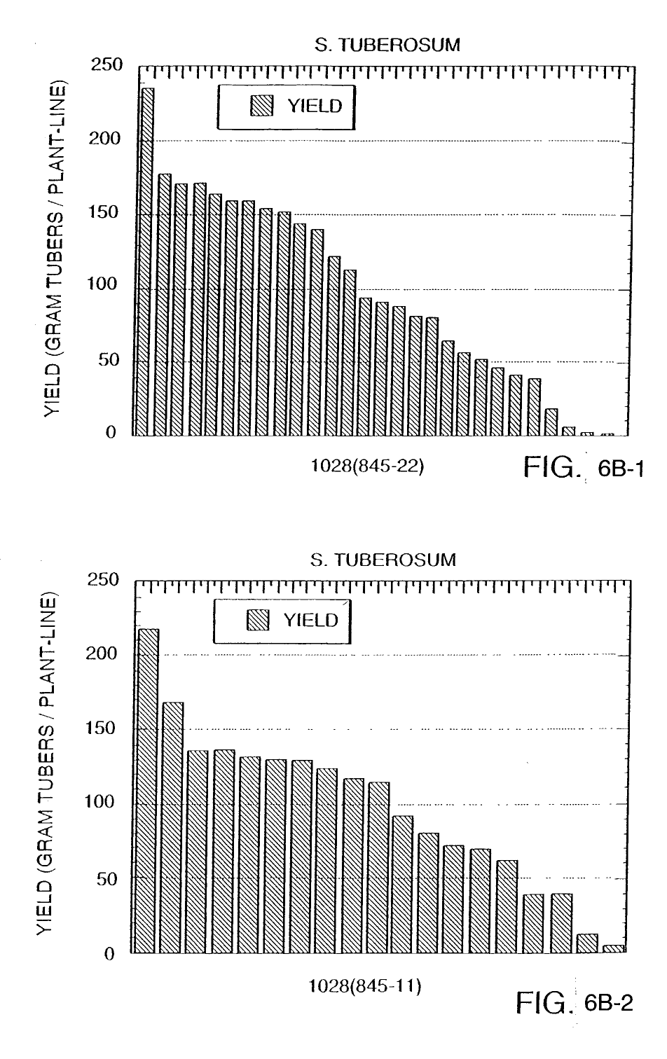

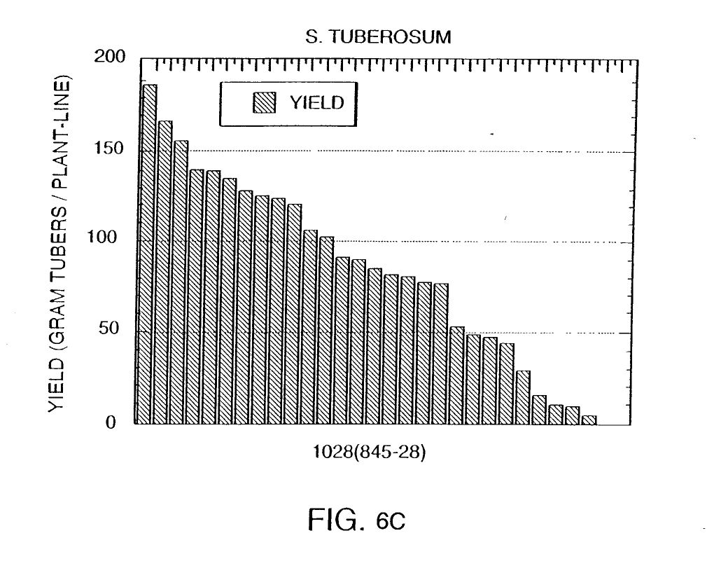

Plants expressing Pat as-trehalase and Pat-TPS simultaneously were generated by retransforming Pat-TPS lines (resistant against kanamycin) with construct pMOG1028, harbouring the Pat as-trehalase construct and a hygromycin resistance marker gene, resulting in genotypes pMOG1028(845-11), pMOG1028(845-22) and pMOG1028(845-28). Plants were grown in the greenhouse and tuber yield was determined (FIG. 6). A number of pMOG1028 transgenic lines yielded significantly more tuber-mass compared to control lines. Individual plants transgenic for both Pat TPS and Pat as-trehalase revealed a varying tuber-yield from almost no yield up to a yield comparable to or higher then the control-lines (FIG. 6).

Construct: PC as-trehalase (pMOG1092)

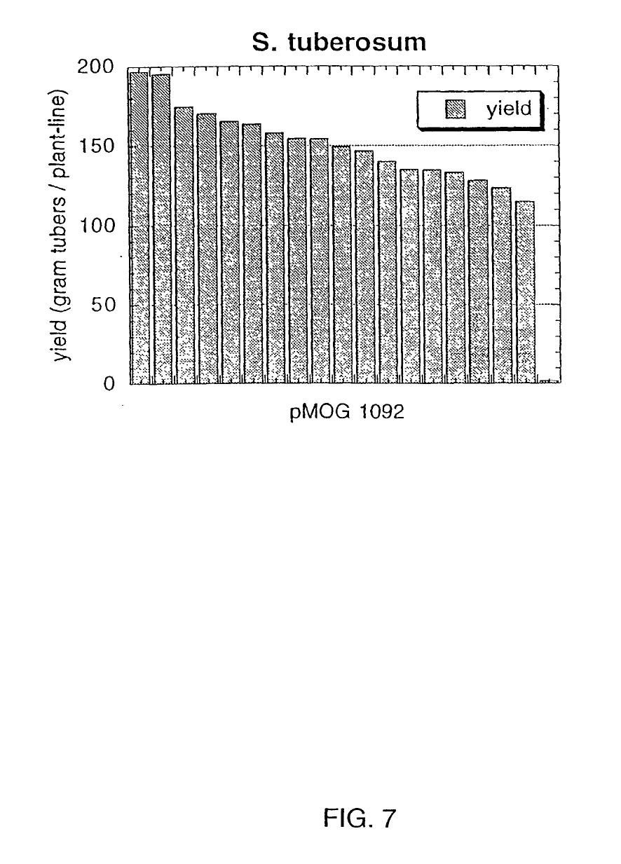

Plants transgenic for pMOG1092 were grown in the greenhouse and tuber-yield was determined. Several lines formed darker-green leaves compared to controls. Tuber-yield was significantly enhanced compared to non-transgenic plants (FIG. 7).

Construct: PC as-trehalase PC-TPS (pMOG 1130)

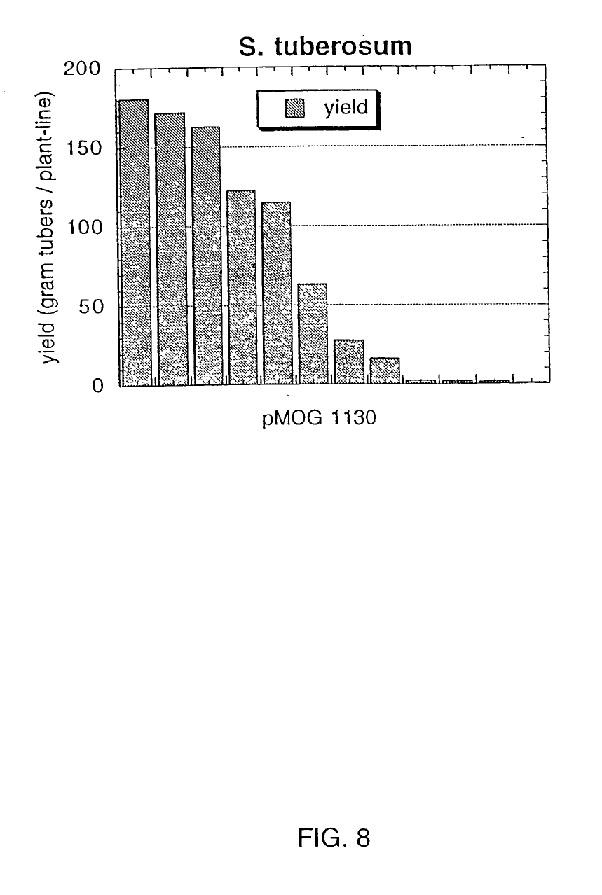

Plants transgenic for pMOG 1130 were grown in the greenhouse and tuber-yield was determined. Several transgenic lines developed small dark-green leaves and severely stunted growth indicating that the phenotypic effects observed when plants are transformed with TPS is more severe when the as-trehalase gene is expressed simultaneously. Tuber-mass yield varied between almost no yield up to significantly more yield compared to control plants (FIG. 8).

### EXAMPLE 7

Overexpression of a Potato Trehalase cDNA in N. Tabacum

Construct: de35S CaMV trehalase (pMOG1078)

Primary tobacco transformants transgenic for pMOG1078 revealed a phenotype different from wild-type tobacco, some transgenics have a dark-green leaf colour and a thicker leaf (the morphology of the leaf is not lancet-shaped) indicating an influence of trehalase gene-expression on plant metabolism. Seeds of selfed primary transformants were sown and selected on kanamycin. The phenotype showed to segregate in a mendelian fashion in the S1 generation.

List of Relevant pMOG### and pVDK### Clones

1. Binary vectors¹

| | |
|---|---|
| pMOG23 | Binary vector (ca. 10 Kb) harboring the NPTII selection |
| | marker |
| pMOG22 | Derivative of pMOG23, the NPTII-gene has been replaced |
| | by the HPT-gene which confers resistance to hygromycine |
| pVDH 275 | Binary vector derived from pMOG23, harbors a |
| | plastocyanin promoter-nos terminator expression cassette. |
| pMOG402 | Derivative of pMOG23, a point-mutation in the NPTII-gene |
| | has been restored, no KpnI restriction site present in the |
| | polylinker |
| pMOG800 | Derivative of pMOG402 with restored KpnI site in |
| | polylinker |

2. TPS/TPP expression constructs

| | |
|---|---|
| pMOG 799 | 35S-TPS-3′nos¹ |
| pMOG 845 | Pat-TPS-3′PotPiII |
| pMOG 1093 | Plastocyanin-TPS-3′nos |
| pMOG 1140 | E8-TPS-3′nos |

3. Trehalase constructs

| | |
|---|---|
| pMOG 1028 | Patatin as-trehalase 3′PotPiII, Hygromycin resistance |
| | marker |
| pMOG 1078 | de35S CaMV amv leader trehalase 3′nos |
| pMOG 1027 | idem with Hyg marker |
| pMOG 1092 | Plastocyanin-as trehalase-3′nos |
| pMOG 1130 | Plastocyanin-as trehalase-3′nos Plastocyanin-TPS-3′nos |
| pMOG 1153 | E8-TPS-3′nos E8-as trehalase-3′PotPiII |
| pMOG 1261 | Tobacco trehalase cDNA fragment |

¹All constructs harbour the NPTII selection marker unless noted otherwise

What is claimed is:

1. A method of modification of the development and/or composition of cells, tissues, or organs in vivo in plants by inhibiting the level of an endogenous trehalase, wherein said cells, tissues, or organs have been genetically altered to comprise a DNA sequence encoding a trehalase inhibitor, wherein said DNA sequence is capable of expressing an RNA that is at least partially complementary to an RNA produced by a DNA sequence encoding the endogenous trehalase, and wherein said modification is other than to increase production or accumulation of trehalose.

2. The method according to claim 1, wherein the DNA sequence encoding the endogenous trehalase comprises a nucleotide sequence encoding an amino acid sequence selected from SEQ ID NO: 4, SEQ ID NO: 6, SEQ ID NO: 8, and SEQ ID NO: 10.

3. The method according to claim 2, wherein the DNA sequence encoding the endogenous trehalase comprises a nucleotide sequence selected from SEQ ID NO: 3, SEQ ID NO: 5, SEQ ID NO: 7, and SEQ ID NO: 9.

4. The method according to claim 1, wherein said modification comprises inhibition of carbon flow in the glycolytic direction.

5. The method according to claim 1, wherein said modification comprises stimulation of photosynthesis.

6. The method according to claim 1, wherein said modification comprises stimulation of sink-related activity.

7. The method according to claim 1, wherein said modification comprises prevention of cold sweetening in potato tuber.

8. The method according to claim 1, wherein said modification comprises inhibition of invertase in beet after harvest.

9. The method according to claim 1, wherein said modification comprises increasing the yield, and wherein said increase in yield is other than the intracellular trehalose content.

10. A method of modification of the development and/or composition of cells, tissues, or organs in vivo in plants by inhibiting the level of an endogenous trehalase, wherein said cells, tissues, or organs have been genetically altered to comprise a DNA sequence encoding a trehalase inhibitor, wherein said DNA sequence comprises a DNA sequence which is identical to a DNA sequence encoding the endogenous trehalase, and wherein said modification is other than to increase production or accumulation of trehalose.

11. The method according to claim 10, wherein the DNA sequence encoding the endogenous trehalase comprises a nucleotide sequence encoding an amino acid sequence selected from SEQ ID NO: 4, SEQ ID NO: 6, SEQ ID NO: 8, and SEQ ID NO: 10.

12. The method according to claim 11, wherein the DNA sequence encoding the endogenous trehalase comprises a nucleotide sequence selected from SEQ ID NO: 3, SEQ ID NO: 5, SEQ ID NO: 7, and SEQ ID NO: 9.

13. The method according to claim 10, wherein said modification comprises inhibition of carbon flow in the glycolytic direction.

14. The method according to claim 10, wherein said modification comprises stimulation of photosynthesis.

15. The method according to claim 10, wherein said modification comprises stimulation of sink-related activity.

16. The method according to claim 10, wherein said modification comprises prevention of cold sweetening in potato tuber.

17. The method according to claim 10, wherein said modification comprises inhibition of invertase in beet after harvest.

18. The method according to claim 10, wherein said modification comprises increasing the yield, and wherein said increase in yield is other than the intracellular trehalose content.
